(19) United States
(12) Patent Application Publication
(10) Pub. No.: US 2008/0317824 A1
(43) Pub. Date: Dec. 25, 2008

(54) Chocolate Coated Spherical Filled Capsules

(75) Inventor: Jochen Wonschik, Brevoerde (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GMBH & CO. KG, HOLZMINDEN (DE)

(21) Appl. No.: 12/063,924

(22) PCT Filed: Aug. 15, 2006
(86) PCT No.: PCT/EP2006/065323

§ 371 Date: Aug. 12, 2008

(30) Foreign Application Priority Data
Aug. 16, 2005 (EP) PCT/EP2005/054019

(51) Int. Cl. A61K 9/00; A23L 1/48; A23P 1/08

(52) U.S. Cl. 424/439; 426/89; 426/103; 426/273

(57) ABSTRACT

Described is a spherical coated capsule comprising a coating-free capsule having (i) a liquid or viscous core (1) and (ii) a solid shell (2) surrounding this core, and a solid chocolate coating (4) surrounding said coating-free capsule, wherein the diameter of the spherical chocolate coated capsule is in the range of 4-20 mm, the solid chocolate coating (4) comprises chocolate in an amount in the range of 20-95% (m/m), based on the total mass of the chocolate coated capsule, the diameter of the coating-free capsule is in the range of 3-10 mm, the thickness of the shell (2) of said coating-free capsule is in the range of 20-200 μm, the ratio of shell thickness to diameter of said coating-free capsule is in the range of 0.004-0.04, the shell (2) of said coating-free capsule contains 70-90% (m/m) gelatine or alginate and 10-30% (m/m) plasticiser, based on the dry mass of said shell, and the core (1) has a flavouring content in the range of 1-100% (m/m), based on the total mass of the core.

The present invention relates to chocolate coated spherical capsules comprising (a) a coating-free capsule having a liquid or viscous core and a solid shell surrounding this core, and (b) a solid chocolate coating surrounding said coating-free capsule; the core of the coating-free capsule (a) comprises a flavouring component, the shell comprises gelatine or alginate and the coating (b) comprises chocolate. The invention also relates to methods for the preparation of such capsules. The chocolate coated spherical capsules according to the invention are suitable for immediate consumption without further processing and can be sucked or chewed in the mouth without the shell being found to be disturbing during consumption, in particular without being sticky or tacky.

Flavourings are used in foods in order to achieve taste impressions. In the sense of the present invention a flavouring is to be understood to be a single substance or a mixture of substances having organoleptic properties. These organoleptic properties include the characteristics of imparting a specific odour or taste or give rise to specific stimuli, which are transmitted via the trigeminus nerve and are thus detected.

Oils, such as, for example, vegetable oils and other triglycerides, which are used as solvents for the flavouring and themselves have a neutral odour and taste are not regarded as flavourings hereinbelow.

If flavourings are to be ingested by a consumer in portions in the form of a liquid, the use of a capsule in which a core of a quantity of liquid containing flavouring is surrounded by a solid shell is a common measure. In this context the following problems and requirements, in particular, must be taken into consideration:

1. Achieving a pleasant feeling in the mouth when consuming gelatine capsules is a technological challenge. In particular, hitherto it has been found to be disadvantageous that the majority of shells of ready-to-consume capsules available commercially are detectable as an unpleasant, rubbery, tough residue. Corresponding observations are made and found to be particularly negative especially in the case of larger filled capsules with diameter of 4 mm or more.
2. On direct consumption, i.e. by placing the capsule directly into the oral cavity, the shell of said capsule in the mouth should preferably (a) not have a disturbing haptic effect, (b) dissolve rapidly and (c) not be sticky or tacky. The core liquid containing flavouring that is to be released or is released should give rise to a sensory effect with a substantial impact in the mouth.
3. A capsule diameter of about 5 mm and more is required for better handling and portioning in the case of direct consumption and a strong flavouring impact by a single capsule in the mouth.

Spherical coating-free capsules having a liquid core and a solid shell surrounding this core are known and capsules having a diameter of more than 4 mm can, for example, be prepared by the rotary die method or, in the case of the capsules having a seamless shell, which are of particular interest, by a drip method using a multi-component nozzle (cf. Bauer, Frömming, Führer; Pharmazeutische Technologie; 1997). This method is also designated a multi-component nozzle method below. In this context (insofar as nothing different results from the context) references to the multi-component nozzle method are also to be understood as references to a multiplicity of related methods for the preparation of seamless coating-free capsules.

For example, in the multi-component nozzle method capsules having a seamless shell are prepared by a drip method. With this method a lipophilic core material and a hot gelatine solution are usually simultaneously pumped through a concentric multi-component nozzle so that they drip into a cold lipophilic cooling liquid, for example vegetable oil. With this method the nozzle can dip directly into the cooling liquid. When they drip in the capsules assume a ball shape (spherical shape) as a result of the surface tensions. As a result of the fall in temperature on contact with the cooling liquid, the gelatine-containing seamless capsule shell solidifies.

U.S. Pat. No. 4,481,157 and U.S. Pat. No. 4,251,195 describe methods and equipment for the continuous preparation of seamless capsules by the multi-component nozzle method where the nozzle dips into the cooling liquid.

WO 03/055587 A1 relates to mononuclearly filled seamless microcapsules comprising a capsule shell made of a hardened capsule material based on an acid polysaccharide, e.g. alginate, and a filling material, which is enclosed on all sides by the capsule shell and which comprises an organoleptically effective quantity of an aroma. These microcapsules can have diameters of up to 5 mm.

However, the larger a coating-free capsule the more difficult it is to achieve a thin, stable shell, since the stability of the coating-free capsule decreases substantially on drying and during transport as the capsule diameter/shell thickness ratio increases. Moreover, the centering of the core and the uniform enclosure of the core by a shell is extremely problematical in the case of large capsules.

U.S. Pat. No. 5,300,305 describes seamless capsules having a diameter of 2-9 mm, which are suitable for immediate consumption and are used to control bad breath. With these capsules, active substances for bad breath control are incorporated in the shell of the capsules on solubility grounds. The capsules are intended to remain in the mouth for a prolonged period so that the active substance influencing the bad breath is able to dissolve from the shell and give rise to a long-lasting effect in the mouth. The shell thickness is in the range of 30 μm to 2 mm. Illustrative embodiments show shell contents of not less than 13% (m/m) and only low plasticiser contents (sorbitol <10%, based on the shell). The capsule contains up to 25% (m/m) flavouring in the core, based on the total mass of the capsule. The capsules are, for example, prepared by a multi-component nozzle process. The capsule shell was developed with a view to slow dissolution in the mouth and the Applicant's experiments have now shown that it proves to be rather hard and annoying when sucked. Accordingly, in particular the adverse sensation in the mouth, that is caused by the shell residues dissolving only slowly in the mouth, is a disadvantage when using the capsule described in U.S. Pat. No. 5,300,305 for an immediate, strong flavouring impression. Moreover, as has been mentioned, the flavouring content in the capsule is restricted to 25% and it is therefore not possible to achieve a strong flavouring impact.

WO 96/29986 describes seamless capsules having a diameter of 2-9 mm, which contain a pharmaceutical active substance against coughing. The capsule shells are 30 μm-500 μm thick and are intended to dissolve within 3.5-5 minutes. The capsule shells contain at least 10% water. A shell thickness to capsule diameter ratio is not given.

A disadvantage of the capsules according to WO 96/29986 is, in particular, the indicated slow rate of dissolution of the shell.

WO 00/51574 describes chewable, soft gelatin capsules having a sheath formed of a mixture of a low bloom and a medium bloom gelatins, a plasticiser, water, and preferably a moisture retention agent to enhance the machinability and integrity of the sheath composition; and a fill of an active material in a carrier liquid.

Since all the above described capsules have an outer shell comprising gelatine, alginate or similar shell materials, they all suffer to some extent from the dissolution properties of the shell material in the oral cavity, causing (a) an unpleasant haptic effect, (b) a comparatively long residence time in the oral cavity during dissolution and (c) a certain stickiness or tackiness.

WO 03/045166 concerns a capsule comprising a core and a coating including at least a film-forming polymer, characterised in that it has a total solubilisation time of its coating not more than 85 s according to a test A. In example 4 a confectionery product for oral hygiene is produced. A seamless gelatine capsule is coated in a sugar-coating pan using maltitol, gum arabic, shellac gum, vegetable oil, titanium dioxide and a menthol-flavouring powder. It was observed that this coated capsule does not leave a skin effect at the end of eating and leaves a powerful aromatic impact in the mouth. The coating composition was not specified in more detail.

U.S. Pat. No. 6,200,603 discloses a coated capsule containing a fill composition, the coated capsule comprising a gelatin shell having water and a plasticiser, wherein the ratio of plasticiser to gelatin in the shell composition ranges from 0.7 to 1.2, preferably from 0.8 to 1.0, and a flavoured coating thereon, wherein each of said flavoured coating and shell material comprises a sugar or sugar substitute adapted to form a stable bond between the shell and the coating and to prevent the coating from drawing water or plasticiser from the gelatin shell.

A breath freshener is provided in the form of a filled gelatin capsule to which is applied a flavoured coating. Such a coated capsule is especially suitable for swallowing prior to rupture of the capsule, whereby the capsule fill is not released until the capsule shell is broken down in the stomach. In a preferred embodiment the composition of the shell material of the coated capsule comprises: gelatin in an amount from 33 to 58% by weight; glycerol in an amount from 16 to 31% by weight; sugar or sugar substitute in an amount from 15 to 30% by weight; and water in an amount up to 15% by weight.

An example for a breath freshener in the form of a filled capsule is given. The capsule consists of 29.8% core materials, 18.6% shell materials and 51.6% by weight of coating materials. The total weight of the capsule materials amounts to 537 mg.

EP 0 778 083 A1 concerns seamless capsules and methods for producing the same.

U.S. Pat. No. 6,238,690 B1 relates to food products containing seamless capsules.

WO 91/17821 A1 relates to microcapsules containing a flavourant embedded in a matrix material.

A primary aim of the present invention was to indicate spherical capsules of the initially mentioned type that are suitable for immediate consumption without further processing and can be sucked or chewed in the mouth without the shell being found to be annoying, in particular without being sticky or tacky, i.e. without sticking to teeth, tongue, gums or palatine. In addition a pleasant mouthfeel should be experienced when consuming the spherical capsule. At least some, but preferably all, the problems and requirements indicated above were to be solved or taken into account. In addition, a method of preparation for the capsules that is practicable on an industrial scale was to be indicated.

According to the invention the above problem is solved by providing a spherical coated capsule comprising

(a) a coating-free capsule having (i) a liquid or viscous core and (ii) a (preferably seamless) solid shell surrounding this core, and
(b) a (preferably seamless) solid chocolate coating surrounding said coating-free capsule, wherein
the diameter of the spherical chocolate coated capsule is in the range of 4-20 mm,
the solid chocolate coating comprises chocolate in an amount in the range of 20-95% (m/m), based on the total mass of the chocolate coated capsule,
the diameter of the coating-free capsule is in the range of 3-10 mm,
the thickness of the shell of said coating-free capsule is in the range of 20-200 μm,
the ratio of shell thickness to diameter of said coating-free capsule is in the range of 0.004-0.04,
the shell of said coating-free capsule contains 70-90% (m/m) gelatine or alginate and 10-30% (m/m) plasticiser, based on the dry mass of said shell, and
the core has a flavouring content in the range of 1-100% (m/m), based on the total mass of the core.

Herein, the “dry mass” of the shell is the mass of the shell after complete removal of water.

The capsules of the present invention can be prepared by a method comprising the following steps:
preparing a coating-free capsule having (i) a liquid or viscous core and (ii) a solid shell surrounding this core,
chocolate coating the coating-free capsule, optionally only after applying an intermediate layer or intermediate layers to the coating-free capsule, and
optionally polishing and/or sealing the chocolate coating.

The preferred capsules of the present invention which comprise a capsule having a seamless solid shell surrounding its liquid or viscous core can be prepared by a method comprising the following steps:
pumping a liquid or viscous core material and a gelatine or alginate-containing curable shell mixture simultaneously through a concentric multi-component nozzle so that they drip into a cooling liquid with the formation of a capsule (i.e. forming a capsule upon immersion into the cooling liquid),
drying said capsule,
chocolate coating the resulting dried capsule (hereinafter also: coating-free capsule), optionally only after applying an intermediate layer or intermediate layers to the dried capsule, and
optionally polishing and/or sealing the chocolate coating.

Preferred embodiments of the present invention are described in the following part of the description and the attached claims.

Throughout the present description a capsule is designated a spherical capsule insofar as the ratio between the largest and the smallest diameter of the capsule is not more than 1.2. This arithmetic mean of the largest and the smallest diameter of the coating-free capsule is designated as the diameter of a coating-free capsule according to the invention.

Surprisingly, it has been found that chocolate coated capsules of the present invention when a sucked or chewed in the mouth provide a sensorial sensation which is not disturbed by the presence of the solid shell (2) of the coating-free capsule. It is assumed that, in particular when the chocolate coating is seamless, there is some kind of surprising (sensorial) interaction between the (seamless) chocolate coating and the shell which makes the consumption of the capsules of the present invention particularly pleasant. Thus, the present invention provides for a new type of chocolate consumable article with hitherto unknown sensorial properties. On consumption of the chocolate coated capsules according to the invention, in particular during biting or chewing thereon, the core liquid passes directly into the mouth which typically gives rise to an immediate flavour impression (flavour burst), especially when chewing or biting causes rupture of the shell of the coating-free capsule (a).

Capsules of the present invention which comprise a capsule having a seamless solid shell surrounding its liquid or viscous core are preferred for technical and sensorial reasons. In particular, a seamless solid shell can be very thin and still be stable. The disturbing sensorial effect of thin capsule shells on the taste sensation during biting, sucking or chewing is particularly low.

Capsules of the present invention which comprise a seamless solid chocolate coating are preferred for sensorial reasons. In addition, capsules having a seamless solid chocolate coating are mechanically more stable than those prepared from two chocolate hemispheres encasing the (seamless) coating-free capsule.

For sensorial reasons it is particularly advantageous if the diameter of the coating-free capsule is in the range of from 4-9 mm, the thickness of the shell is in the range of 30-150 μm and the ratio of shell thickness to coating-free capsule diameter is in the range of from 0.006-0.025. It is most advantageous if the diameter of the coating-free capsule is in the range of 4.5-8.5 mm, the thickness of the shell is in the range of 40-120 μm and the ratio of shell thickness to diameter of the coating-free capsule is in the range of 0.008-0.02.

Preferred embodiments of the coating-free capsule being the inner part of the capsules according to the invention result from the following description, examples, and patent claims. Hereinafter, the emphasis is on seamless coating-free capsules, but the invention is not limited to the use of such seamless capsules.

Unless indicated otherwise, all percentages and ratios given are by weight and all conditions (e.g. state of aggregation) refer to 20° C.

### Shell Thickness:

For rapid dissolution of the shell of a coating-free capsule (as part of the capsule of the present invention) in the mouth the shell thickness should be as small as possible. For a constant core/shell mass ratio the shell thickness increases substantially with increasing diameter of the coating-free capsule.

The coating-free capsules have a shell thickness of only 20 μm-200 μm despite the large diameter of the coating-free capsule of 3-10 mm. In these capsules the ratio of shell thickness to capsule diameter is in the range of 0.004-0.04. These data relate to the dried capsule. The coating-free capsule diameter can be determined using a micrometer screw. For determination of the shell thickness a cross-section of the coating-free capsule is prepared. The thickness of the shell can be determined via a microscope with image processing. To this end the thickness of the coating-free capsule shell is measured at various points in the latter and the mathematical mean for the shell thickness is determined.

With the composition of the shell selected in accordance with the invention (in this context see below) high process and transport stability, adequate elasticity, good biteability and a sufficiently high dissolving ability in the mouth are ensured.

### Composition of the Shell:

### Viscosity of the Shell Mixture/Gel Point:

When shaping a seamless coating-free capsule (as part of the capsule of the present invention) by means of a multi-component nozzle method particular attention has to be paid to the viscosity and the gelling characteristics of the curable shell mixture. Too low a viscosity or too low a gel point prevent a capsule shell that is sufficiently solid in the wet state. Corresponding capsules would be mechanically destroyed by the further processing steps, such as, for example, centrifuging. Too high a viscosity and too high a gel point, on the other hand, prevent correct capsule shaping and, moreover, give rise to undesired, substantial satellite formation.

The viscosity of preferred shell mixtures for the preparation of seamless coating-free capsules was determined using a CVO 120 rheometer (Bohlin Instruments GmbH, Pforzheim). The measurement system used was a plate-plate system with a plate diameter of 50 mm. The measurements were carried out using rotation. The shear rate was 50 s⁻¹; the gap was set at 500 μm. The measurements were carried out isothermally; the temperature was 80° C.

At 80° C. preferred shell mixtures have a viscosity in the range of 30 mPas to 300 mPas, preferably of 40 mPas to 150 mPas, and particularly preferentially of 50 mPas to 90 mPas.

The gel point of preferred shell mixtures for the preparation of seamless coating-free capsules was likewise determined using a CVO 120 rheometer (Bohlin Instruments GmbH, Pforzheim). The measurement system used was a plate-plate system with a plate diameter of 50 mm. The measurements were carried out using oscillation. The frequency was constant at 1 Hz, the gap was set at 500 μm and the temperature was lowered from 80° C. to 10° C. with a gradient of 5° C./min. The temperature at which the viscosity or memory module G′ is equal to the modulus of elasticity or loss module G″ was read off as the gel point, the sol/gel transition point (Thomas Mezger, Das Rheologie Handbuch, 2000).

The gel points of preferred shell mixtures are between 15° C. and 60° C., preferably between 20° C. and 40° C. and particularly preferentially between 25° C. and 35° C.

### Gelatine:

The shell of the (preferably seamless) coating-free capsules (as part of the capsule according to the present invention) contains gelatine or alginate and plasticiser, preference is given to gelatine. The grade and amount of the gelatine and of the plasticiser have an effect on the solubility kinetics of the shell in the mouth.

For shaping of the seamless coating-free capsule, aqueous solutions containing 10-50% (m/m), preferably 15-40% (m/m) and particularly preferentially 15-35% (m/m) gelatine are preferably used for the shell.

The gelatine used in the capsules according to the invention is in many cases obtained by partial hydrolysis of collagen-containing material from animals, such as, for example, pigs, cattle, fish or poultry. Type A gelatine is obtained by acid digestion, usually of pig or fish skins, whilst type B gelatine is obtained by alkaline digestion, usually of cattle bones and skins.

The term Bloom is used to characterise the gel strength of gelatine. In the determination of the Bloom value a stamp of a Bloom gelometer or texture analyser 12.7 mm (0.5 inch) in diameter is pressed 4 mm deep into a 6.67% gelatine gel that has been aged for 18 hours at 10° C. prior to the measurement. The result is given in “Bloom”, corresponding to the weight in grams that is loaded onto the stamp in order to achieve the impression depth (see Schormüller, Handbuch der Lebensmittelchemie, Volume III, 1968 and British Standard Method for Sampling and Testing Gelatine (BS757; 1975)).

A gelatine having a Bloom value of more than 200, particularly preferentially having a Bloom value of 240-300, and specifically preferably a Type A gelatine is preferably used for the preparation of coating-free capsules according to the invention. By this means adequate stability of the shell is made possible during the preparation of the coating-free capsule and during transport, despite the small thickness of the capsule shell.

Gelatine grades that have been obtained from cattle, poultry or fish are also suitable for the preparation of the capsule according to the invention. In this context, in any event, as already mentioned, care must be taken that the viscosity and the gelling characteristics are correctly adjusted. Fish gelatines that can be used are both grades from cold water fish and grades from warm water fish. Mixtures of different gelatine grades can also be used.

Achieving high process stability of a coating-free capsule coupled with rapid solubility of the capsule shell in the mouth is a particular technical problem. On the one hand, it is advantageous, specifically for the process for preparation of the capsule and for storage, to select a shell formulation that gives the coating-free capsule a particularly high mechanical strength and rapidly forms a solid gel that after drying is as hard as possible and absorbs little water. On the other hand, for a good sensation in the mouth on consumption of the final coated capsule it is advantageous if the shell absorbs water rapidly, is soft and flexible and dissolves quickly.

It has been found that this particular technical problem can be solved by the use of a mixture of a hydrolysed gelatine with a Bloom value of 0 and a high-Bloom gelatine with a Bloom value of 200 and above (preferably a Bloom value in the range of 240-300). Presumably the high-Bloom gelatine forms a solid network here that is important for the process stability. The hydrolysed 0-Bloom gelatine presumably occupies spaces in this network and in the mouth leads to a more rapid absorption of water and thus solubility of the entire shell.

Hydrolysed 0-Bloom gelatine has no gelling power and is readily soluble in water at 20° C. In this 0-Bloom gelatine the polypeptide chains have been very substantially decomposed by acid or enzymatic hydrolysis. To date it is therefore also not used for the formation of coating-free capsule shells but is used only, for example, as a nutrient (protein source), as an emulsifier or also for clarifying wine.

Shell mixtures consisting of (a) hydrolysed 0-Bloom gelatine, that has been obtained from any desired species of animal, with (b) gelatine that has a Bloom value of ≧200 are preferably suitable for solving the particular technical problem, the proportion of hydrolysed 0-Bloom gelatine preferably being in the range of 0.5-90% (m/m), based on the dry mass of the shell.

Here the gel point of the high-Bloom gelatine constituent is the decisive factor determining the gel point of the mixtures (see the appended table “Gel points”).

The flexibility of a film produced from a shell mixture is, moreover, surprisingly high if the mixture contains 0-Bloom gelatine.

A further possibility for solving the abovementioned particular technical problem consists in the mixture of certain low-Bloom (Bloom value<200) fish gelatines with high-Bloom gelatine (Bloom value≧200).

The lower the Bloom value of a gelatine, the lower are, in general, the gel point, the viscosity and the mechanical stability of the moist solidified gel. Mixtures of gelatine having a low Bloom value and a medium Bloom value in order to achieve an improved solubility of the coating-free capsule shell in the mouth are known. U.S. Pat. No. 6,258,380 describes shells of this type.

It has now been found that when fish gelatine is used as the gelatine fraction having a low Bloom value (<200) and at the same time a high-Bloom gelatine having a Bloom value of 200 and above is used, a further improved solubility of the coating-free capsule in the mouth can be achieved. This is presumably effected by the lower gel point of fish gelatines (below 28° C.) compared with pig, cattle and poultry gelatines (approximately 28-40° C.).

Fish gelatine grades with a gel point of <20° C. and grades that are prepared from cold water fish, for example cod, are particularly suitable since their gel points at approximately 10-20° C. are even below those of gelatines from warm water fish (gel point approximately 20-28° C.), such as, for example, from carp. In this context the cold water fish include all species of fish that live predominately in waters at temperatures of 18° C. and below. Research by Choi and Regenstein (Journal of Food Science Vol. 65, No. 2, 2000) and the Applicant's research (see appended table “Gel points”) confirm the lower gel points of fish gelatines compared with pig, cattle and chicken gelatines with approximately comparable Bloom values.

Presumably the protein composition is of importance for the lower gel points in fish gelatine grades. The proportions of the amino acids proline and hydroxyproline are considerably lower in the case of fish gelatines, and specifically especially in the case of cold water fish gelatines, compared with pig, cattle and poultry gelatines. Hydroxyproline and proline play an important role in the cross-linking of the protein helices with one another. Presumably folding of the helices takes place in water, water can be embedded and the solubility rises. The temperature at which this folding takes place depends on the hydroxyproline content and proline content. The lower the content, the lower the temperature at which the gelatine goes into solution.

A low gel temperature and a low solubility temperature is advantageous for good solubility in the mouth.

As a rule it is not possible to achieve adequate process stability of the coating-free capsule by using fish gelatine having a Bloom value of less than 200 as the only type of gelatine in the shell. The gel strength of the coating-free capsule shells that are still moist is frequently not adequate for further processing. The coating-free capsules are frequently mechanically too unstable.

On the other hand, the particular technical problem is solved by the admixture of such a gelatine, as readily soluble filler, to a high-Bloom gelatine that is intended to form a process-stable skeleton. In this context mixtures of fish gelatines with Bloom values of below 200 and high-Bloom pig, cattle or poultry gelatines with a Bloom value of over 200 have proved advantageous. Fish gelatine (below 200 Bloom) contents of 0.5-50% (m/m), based on the dry mass of the shell, are preferred. In this context gelatine grades from cold water fish are particularly preferred.

Here it is the gel point of the high-Bloom gelatine fraction that is the decisive factor determining the gel point of the mixtures (see the appended table “gel points”).

### Plasticisers:

Plasticisers that can be used in the preparation of the shell are, in particular, polyols, such as, for example, sorbitol, glycerol, propylene glycol, lactitol, hydrated hydrolysed starches and trehalose. Plasticiser fractions improve the consumption characteristics of a capsule in that they reduce the hardness of the shell of the coating-free capsule and improve the solubility in the mouth. Moreover, plasticisers promote the flexibility of the shell and thus the stability during drying of the coating-free capsule and during transport.

Preferred plasticiser contents are not more than 30% (m/m) based on the dry mass of the shell. Higher amounts of plasticiser make drying of the coating-free capsules more difficult and also make it necessary to use packaging that excludes atmospheric humidity.

Plasticisers are preferably used in the shell in a proportion of 10-30% (m/m), particularly preferentially of 15-25% (m/m), based on the dry mass of the shell. The plasticiser preferably comprises one or more polyols, preferably selected from the group which consists of glycerol, propylene glycol, sorbitol and maltitol. Glycerol is the preferred plasticiser.

Plasticiser contents of over 30% (m/m) make drying of coating-free capsules (as part of the capsules according to the present invention) more difficult and frequently make it necessary to use anticaking agents, such as silica.

Plasticiser contents of less than 10% (m/m) allow the capsule shell of a coating-free capsule to become increasingly brittle.

Experiments have shown that, in the case of sorbitol, contents of more than 15% (m/m) in the shell can already give rise to problems with capsule drying and would then make the undesired use of an anticaking agent necessary.

### Further (Optional) Constituents of the Capsule Shell:

### Sweeteners/Colourants/Acids/Water/Breath Control Actives/Flavourings/—Water Soluble Nutraceuticals:

In addition to gelatine and plasticiser, the shell of the coating-free capsule (as part of the capsule of the present invention) can contain sweeteners, such as, for example, sucralose, aspartame, acesulfame, K or Na saccharine, thaumatin, neohesperidin, or mixtures thereof, as well as water-soluble food colourants.

In addition to the materials mentioned above, the shell of a coating-free capsule can contain acids, in particular of the monoacid, diacid or triacid type, preferably citric acid, fumaric acid, malic acid, succinic acid, tartaric acid, adipic acid, lactic acid, ascorbic acid, or acetic acid. The use of at least one such acid makes it possible, in particular, to ensure microbiological stability of the shell of the coating-free capsule and to tailor its sensory properties. In addition the physicochemical properties can be adjusted (pH, solubility).

The coating-free capsules are typically dried during the production process. During this operation a certain residual amount of water remains bound in the gelatine network. Depending on the ambient moisture content, a water content will be established in the coating-free capsule shell in equilibrium. At 20° C. and 50% relative atmospheric humidity, the equilibrium moisture content of typical coating-free capsules (as part of the capsules according to the invention) is in the range of approximately 8-10% (m/m) water, based on the total mass of the coating-free capsule shell.

Breath control actives, suitable for inclusion in the shell of the coating-free capsules are quaternary compounds such as pyridinium salts (e.g., cetyl pyridinium chloride), other cationic materials such as chlorhexidine salts, zinc salts, salts such as sodium laurate, chlorophyll, triclosan, copper compounds such as copper gluconate or copper-chlorophyll-extract.

The optionally included one or more flavourings in the shell (mixture) may independently from each other be the same or different as the one or more flavourings included in the core liquid (see below). In those cases in which it is not possible to achieve the desired flavour profile with one or more flavourings in the core liquid alone, such as may be the case when a certain and relatively high amount of one or more hydrophilic flavouring substances which are not sufficiently soluble in the lipophilic core liquid are required, one or more flavourings included in the core liquid preferably differ from those in the shell (mixture). By incorporating such one or more hydrophilic flavouring substances (mainly or entirely) into the shell (mixture) the desired flavour profile may be achieved in the way that one or more flavourings in the liquid core and one or more flavourings in the shell (mixture) thereby complementing one another.

One or more of such hydrophilic flavouring substances, which may advantageously be incorporated into the shell (mixture), are for example selected from the group consisting of aqueous (plant) extracts, in the present case preferably coffee-extracts, cinnamon-extracts, thyme-extracts, anise-extracts, clove-extracts, cocoa-extracts or tea-extracts. Preferred are aqueous coffee-extracts, which preferably contain caffeine. Coffee-extracts may be obtained form freshly brewed or roast and ground coffee, which preferably can be of the roasted, sweet, cocoa-like, caramel-like or black roasted type, etc.

The shell (mixture) may contain one or more substances or one or more substance mixtures, which are active in nutritional physiology (water soluble nutraceuticals). Examples for these substances are: vitamin C (ascorbic acid), amino acids, caffeine, capsaicin, etc.

### Hydrocolloids/Gellan Gum:

Additions of hydrocolloids to gelatine influence the solubility and thus the absorption of water as well as the temperature stability of the gels formed.

The hydrocolloid gellan gum, in particular, may be used as an admixture to the gelatine in a shell material mixture for the preparation of a coating-free capsule according to the invention. Gellan gum is a gel-forming polysaccharide that is prepared by fermentation with the aid of microorganisms.

U.S. Pat. No. 4,517,216 already describes mixtures of gelatine and gellan gum. By means of a gellan gum content of 16-83%, based on the sum of the gellan gum amount and gelatine amount, a high gel strength of the shell is achieved as a result of a synergistic effect. It is also described that only the deacylated and partially deacylated forms of gellan gum give rise to this effect, but not the native gellan gum.

If the multi-component nozzle method with immersed nozzle is to be used for the preparation of a seamless coating-free capsule for use according to the invention, it is essential when using gellan gum in the shell material to make the correct choice of the gellan gum type and the gellan gum amount so that the shell does not solidify even before formation of the coating-free capsule has been completed. Moreover, the viscosity of the shell solution must not be too high.

In order to achieve an increased temperature stability, gellan gum is advantageously added to the shell mixture for the preparation of a coating-free capsule according to the invention; as a result the softening temperature of the shell increases considerably and the gelling temperature of the mixture is also considerably increased. For the preparation of seamless coating-free capsules for use according to the invention by the multi-component nozzle method with immersed nozzle, the gelling temperature should not be above 50° C. and the viscosity of the shell solution at 80° C. should not be above 300 mPas; otherwise formation of the coating-free capsule is made more difficult or is not achievable. Therefore, the gellan gum type and amount must be selected in a particularly targeted manner.

There are high-acylated and low-acylated gellan gum grades. A low-acylated gellan gum, preferably the KELCOGEL F grade from Kelco, a division of Merck & Co, is preferably used for the preparation of coating-free capsules according to the invention. Hard, transparent gels can be obtained using low-acylated gellan gum grades.

The preparation of coating-free capsules used in the present invention by the multi-component nozzle process is problematical with a high-acylated gellan gum grade, such as, for example, KELCOGEL LT100 from Kelco, since during capsule shaping the coating-free capsules do not release from the coaxial nozzle without disturbance because of high elasticity of the shell. Moreover, undesired turbid and very highly elastic soft gels are produced.

In a preferred aqueous shell mixture (shell solution) for the preparation of a seamless coating-free capsule for use according to the invention gelatine, with a content of >15% (m/m), based on the total mass of the shell solution, makes up the major proportion of hydrocolloids used in the total mass. In addition, if used at all, gellan gum is used in a proportion of at most 0.6% (m/m), preferably a proportion in the range between 0.2 and 0.5% (m/m).

Higher proportions of gellan gum substantially increase the viscosity of the shell solution during capsule shaping and substantially reduce the solubility of the dried coating-free capsule shell in the mouth, which is not desired.

Lower proportions of gellan gum have no particular effect with regard to improved temperature stability of the dried coating-free capsule.

The presence of gellan gum leads to the formation of a solid network in the shell of a coating-free capsule according to the invention, which solid network as a rule does not dissolve in the moist state, even at 40-60° C. This network should make up only a relatively small proportion of the shell as a strengthening element. The fractions of non-crosslinked gelatine and further additives such as, for example, plasticisers, should, on the other hand, dissolve particularly rapidly in the mouth.

Coating-free capsules (as part of the capsules according to the invention) that contain gellan gum in a range of 0.4-3% (m/m), preferably of 0.8-2% (m/m), based on the dry mass of the shell are preferred. A preferred mass ratio of gellan gum to gelatine in the range of 1:23 to 1:230, preferably of 1:35 to 1:115 is obtained with gelatine contents of 70-90% (m/m), based on the dry mass of the shell.

The gelatine fraction in these coating-free capsules can, in particular, also contain fractions of 0-Bloom gelatine and/or low-Bloom fish gelatine (in this context see above).

### Core Liquid:

When in a process according to the present invention a seamless coating-free capsule is prepared by the multi-component nozzle method the core liquid is hydrophobic and able to form a two-phase system with aqueous solutions.

Generally, the core liquid in the coating-free capsule (seamless or not) can be liquid or viscous, up to paste-like.

On consumption of the chocolate coated capsules according to the invention (capsule diameter in the range of 4-20 mm) a relatively large amount of core liquid passes directly into the mouth. This typically gives rise to an immediate flavour impression, the impact of said flavour impression can be adjusted by type and/or amount of flavouring in the core liquid.

Examples of flavourings which may be a constituent of the core liquid: aliphatic esters (saturated and unsaturated), for example ethyl butyrate, allyl capronate; aromatic esters, for example benzyl acetate, methyl salicylate; organic aliphatic acids (saturated and unsaturated), for example butyric acid, ethanoic acid, hexanoic acid; organic aromatic acids; aliphatic alcohols (saturated and unsaturated), for example ethanol, propylene glycol, octenol; cyclic alcohols, for example menthol; aromatic alcohols, for example benzyl alcohol; aliphatic aldehydes (saturated and unsaturated), for example acetaldehyde, nonadienal; aromatic aldehydes, for example benzaldehyde, vanillin; ketones, for example menthone; cyclic ethers, for example 4-hydroxy-5-methylfuranone; aromatic ethers, for example p-methoxybenzaldehyde; guaiacol; phenolic ethers, for example methoxyvinyl phenol; acetals, for example acetaldehyde diethyl acetal; lactones, for example gamma-decalactone; terpenes, for example, limonene, linalool, terpinene, terpineol, citral (geranial and neral); sulfides, for example dimethyl sulfide; thiols, for example methylfuran thiol; disulfides, for example difurfuryl disulfide; pyrazines, for example methylpyrazine, acetyl pyrazine.

The flavourings used in the context of the present invention may comprise natural flavours, artificial flavours, and mixtures thereof.

Flavourings from the series comprising peppermint oils, spearmint oils, eucalyptus oils, wintergreen oils, cinnamon oils, cassia oils, aniseed oils, bitter almond oils, clove oils, parsley seed oils, citrus oils (e.g. oils of orange, lemon, grapefruit, lime), vanilla (extracts), fruity flavouring compositions having tastes oriented towards, for example, apple, pear, peach, grape, strawberry, raspberry, cherry, orange, lemon, grapefruit, lime or pineapple are preferably used.

Individual preferred one or more cooling agents for use within the context of the present invention, in particular as part of the core liquid, are independently from each other selected from the group consisting of: l-menthol, d-menthol, racemic menthol, menthone glycerol acetal (trade name: Frescolat® MGA), menthyl lactate (trade name: Frescolat® ML; menthyl lactate is preferably l-menthyl lactate, especially l-menthyl l-lactate), substituted menthyl-3-carboxamides (e.g. menthyl-3-carboxylic acid N-ethylamide), 2-isopropyl-N-2,3-trimethylbutanamide, substituted cyclohexanecarboxamides, 3-menthoxypropane-1,2-diol, 2-hydroxyethyl menthyl carbonate, 2-hydroxypropyl menthyl carbonate, N-acetylglycine menthyl ester, isopulegol, hydroxycarboxylic acid menthyl esters (e.g. menthyl 3-hydroxybutyrate), monomenthyl succinate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl-3,6-di- and trioxaalkanoates, 3-menthyl methoxyacetate and icilin.

Preferred cooling agents are l-menthol, d-menthol, racemic menthol, menthone glycerol acetal (trade name: Frescolat® MGA), menthyl lactate (preferably l-menthyl lactate, especially l-menthyl l-lactate (trade name: Frescolat® ML)), substituted menthyl-3-carboxamides (e.g. menthyl-3-carboxylic acid N-ethylamide), 2-isopropyl-N-2,3-trimethylbutanamide, substituted cyclohexanecarboxamides, 3-menthoxypropane-1,2-diol, 2-hydroxyethyl menthyl carbonate, 2-hydroxypropyl menthyl carbonate and isopulegol.

Particularly preferred cooling agents are l-menthol, racemic menthol, menthone glycerol acetal (trade name: Frescolat® MGA), menthyl lactate (preferably l-menthyl lactate, especially l-menthyl l-lactate (trade name: Frescolat® ML)), 3-menthoxypropane-1,2-diol, 2-hydroxyethyl menthyl carbonate and 2-hydroxy-propyl menthyl carbonate.

In addition, suitable individual substances as part of the flavourings are those having a cooling refreshing effect in the throat or in the oral or nasal cavity. Those which may be mentioned, by way of example, are menthol, menthone, carboxamides, menthol acetate, menthol methyl ether, methone acetals, menthol carbonates, menthol succinates, 1,8-cineol (eucalyptol), carvone, alpha-terpineol, thymol, methyl salicylate, 2′-hydroxypropiophenone.

In another preferred embodiment one or more flavourings of the core liquid are independently from each other selected from the following flavour directions: amaretto, anisette, brandy, butterscotch, rum, cappuccino, mint, cinnamon, cinnamon almond, creme de menthe, grand marnier, peppermint, pistachio, chamomile, chocolate, cinnamon spice, caramel, cocoa, lavender, maple, milk (in all forms), cream, butter, vanilla, French vanilla, Irish creme, Kahlua, lemon, hazelnut, almond, pecan, lavender, macadamia nut, orange, orange leaf, peach, apple, strawberry, grape, raspberry, cherry, other fruit flavours, including mixtures thereof.

Preferred flavourings of the core liquid comprise those capable of delivering vanilla, French vanilla, vanilla nut, coffee, hazelnut, Irish creme, amaretto, rum, caramel and almond flavours. In a preferred embodiment of the present invention, preferred flavourings are flavourings imparting a coffee or coffee-like flavour, preferably chosen from regular coffee, cappuccino, latte macchiato, mocha coffee or espresso. Coffee-like flavourings include for example vanilla, butterscotch, almond, lemon or strawberry flavoured cappuccino, cinnamon flavoured coffee or caramel, cocoa or hazelnut flavoured latte macchiato and the like.

Preferably the (coffee) flavourings comprise 1, 2, 3, 4, 5, 6, 7, 8, 9, 10 or more of the following compounds: 2,3-methylfuranthiol; furfurylthiol; furfuryl thioacetate; difurfuryl disulfide; methane thiol; 3-methyl-2-buten-1-thiol (prenylthiol); 3,3-methylthiobutyl formate; 3-methylthiopropanal; o-methylthiophenol (thioguaiacol); 2,6-dimethoxy phenol; 2-acetyl-2-thiazoline (acetylthiazoline); 2-acetylthiazol; 2-methylbutanal; 3-methylbutanal; 2,3-butandione; 2,3-methylethyl pyrazine; 2,3-methoxymethylpyrazine; 3,5,2-methyldiethyl pyrazine; 2-ethyl-3,5-dimethyl pyrazine; 2-ethyl-3,6-dimethylpyrazine; 2-ethyl-3,5-dimethylpyrazine; 3-ethyl-2,5-dimethylpyrazine; 2-methoxy-3-(1-methylpropyl) pyrazine (2,3-methoxyisobutyl pyrazine); 4-vinylguaiacol; 4-ethylguaiacol; 2,5-dimethyl-4-hydroxy-3(2H)-furanone (dimethylhydroxyfuranone-2,5,4,3); 4-methyl-5-ethyl-3-hydroxy-2(5H)-furanone (methylethylhydroxyfuranone-4,5,3,2); acetaldehyde; propionaldehyde; beta-damascenone; 2,3-pentandione; ethyl acetate; acetic acid; caproic acid and butyric acid.

### Flavouring Content in the Liquid or Viscous Core:

The flavouring content in the core liquid depends, in particular, on the capsule size and the flavouring intensity and according to the invention ranges from 1-100%, based on the total mass of the liquid or viscous core. However, a flavouring content in the liquid or viscous core in the range of 5-90% (m/m), preferably 15-70% (m/m), based on the total mass of the liquid or viscous core, is preferred.

### Sweeteners in the Liquid or Viscous Core (Optional):

Sweeteners can also be added to the core liquid of a capsule according to the invention, with the use of solubilising agents if appropriate. Since, in accordance with the intention, the core liquid comes into direct contact with the teeth in the mouth, it is advantageous if the core liquid does not exert a pH-lowering action. Otherwise it would not be possible to preclude damage to the enamel.

In the Applicant's research it has now been found that thaumatin, neohesperidine and miraculin (as well as mixtures thereof) are particularly suitable as sweeteners in the core liquid and do not have an adverse effect on the pH value. On solubility grounds thaumatin is particularly preferred.

It has also been found that, on the other hand, other sweeteners that in principle are suitable for use, such as, for example, saccharinic acid or acesulfame K lower the pH value of the aqueous phase and therefore should not be used in relatively high concentrations if the change in the pH in the mouth is to remain tolerable.

### Oils in the Liquid or Viscous Core:

The oils concerned are preferably fatty oils, in particular, triglycerides, and especially triglycerides of caprylic acid and capric acid, mixtures of triglycerides of the (vegetable) oil type, apricot kernel oil, avocado oil, babassu oil, cottonseed oil, borage oil, thistle oil, hemp oil, hazelnut oil, corn oil, jojoba oil, cherry kernel oil, linseed oil, maize germ oil, macadamia nut oil, almond oil, evening primrose oil, olive oil, pecan nut oil, peach kernel oil, peanut oil, pistachio nut oil, rape-seed oil, rice germ oil, castor oil, safflower seed oil, sesame seed oil, soybean oil, sunflower seed oil, grape-seed oil, wheat germ oil, edible mineral oils and edible silicone oils. Preferred oils for diluting the flavourings used are, in particular, fractionated coconut oils which contain mainly C6-C8 fatty acid radicals. These oils are characterised by their neutral taste and by their good stability to oxidation.

### Further Constituents of the Liquid or Viscous Core (Optional):

Colouring substances, vitamins (e.g. ascorbic acid, vitamin E), and/or vegetable extracts can be added to the core liquid.

The liquid or viscous core may contain substances or substance mixtures, which are active in nutritional physiology (nutraceuticals). The following might be named by way of example: panthenol, pantothenic acid, essential fatty acids, vitamin A and derivatives, carotenes, vitamin C (ascorbic acid), vitamin E (tocopherol) and derivatives, vitamins of the B and D series, such as vitamin B₆ (nicotinamide), vitamin B₁₂, vitamin D₁, vitamin D₃, vitamin F, folic acid, biotin, amino acids, oil soluble compounds of the elements magnesium, silicon, phosphorus, calcium, manganese, iron or copper, coenzyme Q10, unsaturated fatty acids, 3-fatty acids, polyunsaturated fatty acids, γ-linolenic acid, oleic acid, eicosapentaenoic acid, docosahexaenoic acid and derivatives thereof, bisabolene, chloramphenicol, caffeine, capsaicin, prostaglandins, thymol, camphor, gamma-oryzanol, salmon oil, oil soluble or oil miscible extracts, concretes or residues of plant and animal origin.

### Actives in the Liquid or Viscous Core (Optional):

Antitussive actives can be added and include e.g. dextromethorphan, chlophedianol, carbetapentane, caramiphen, nosciapine, diphenylhydramine, codeine, hydrocodone, hydromorphone, fominoben and benzonatate.

Oral anesthetic actives can be added and include e.g. phenol, lidocaine, dyclonine, benzocaine, menthol, salicyl alcohol and hexylresorcinol.

### Further Properties of the Coating-Free Capsule:

The following properties apply for the (preferably seamless) coating-free capsules before coating.

### Hardness:

The coating-free capsules used in the present invention preferably have a hardness of 1000-4000 g. Coating-free capsules which, for example, have a hardness of 1500-3500 g for a diameter of 5 mm are preferred.

Harder coating-free capsules usually give rise to an unpleasant sensation in the mouth; softer capsules give rise to difficulties during transport since they are not mechanically stable.

In this context the hardness of the coating-free capsules is determined using a texture analyser, for example using a TA-XT2i from Stable Micro Systems. With this method a stamp having a diameter of 2 mm is lowered at a constant forward speed of 0.5 mm/sec onto a coating-free capsule until the shell of this capsule breaks. The hardness of the coating-free capsule is designated as the weight in g that bears on the coating-free capsule at the breaking point.

### Dissolving Abilities:

The shell of preferred coating-free capsules (as part of the capsules according to the invention) dissolves in the mouth in less than 60 seconds, preferably in less than 45 seconds. The rate of dissolution can be determined by sensory means, the mechanical effects on moving the coating-free capsule in the mouth also being taken into account.

### Chocolate Coating:

The (preferably seamless) chocolate coating (material) according to the present invention is based on a chocolate product in its broadest meaning. The chocolate coating (material) may for example comprise or consist of the following chocolate products: cocoa (powder), dark chocolate, couverture, semi-sweet chocolate, bittersweet chocolate, milk chocolate, family milk chocolate, white chocolate, chocolate a la taza or chocolate familiar a la taza. The chocolate coating (material) may comprise sugar(s) or be sugar-free. The chocolate coating (material) may further comprise additives useful in the manufacturing process, e.g. edible binder, water or (aqueous) ethanol.

In particular definitions for certain chocolate products, especially those for cocoa (powder), chocolate, milk chocolate, family milk chocolate and white chocolate, are as outlined in the Directive 2000/36/EC of the European Parliament and the Council of the European Community of 23 Jun. 2000 relating to cocoa and chocolate products intended for human consumption (“Chocolate Directive”, published on Mar. 8, 2000 in Official Journal EC 2000, L 197, p. 19-25). It should be mentioned that Directive 2000/36/EC allows the addition of vegetable fats other than cocoa butter to chocolate products, such as cocoa butter equivalents, up to a maximum of 5%. Vegetable fats other than cocoa butter may be hardened or unhardened vegetable fats, mainly these are cocoa butter equivalents, cocoa butter substitutes, cocoa butter extenders or cocoa butter replacers (see also Eur. J. Lipid. Sci. Technol. 2003, 105, 32-42). Accordingly, the chocolate coating of the present invention may include such ingredients.

Preferred chocolate coating materials according to the present invention may be based on the following chocolate products:

### 1. Cocoa Powder, Cocoa

“Cocoa powder”, cocoa designate the product obtained by converting into powder cocoa beans which have been cleaned, shelled and roasted, and which contains not less than 20% cocoa butter, calculated according to the weight of the dry matter, and not more than 9% water.

“Fat-reduced cocoa, fat-reduced cocoa powder” designate cocoa powder containing less than 20% cocoa butter, calculated according to the weight of the dry matter.

“Powdered chocolate, chocolate in powder” designate the product consisting of a mixture of cocoa powder and sugars, containing not less than 32% cocoa powder.

“Drinking chocolate, sweetened cocoa”, sweetened cocoa powder designate the product consisting of a mixture of cocoa powder and sugars, containing not less than 25% cocoa powder.

### 2. Chocolate

“Chocolate” designates the product obtained from cocoa products and sugars which contains not less than 35% total dry cocoa solids, including not less than 18% cocoa butter and not less than 14% of dry non-fat cocoa solids.

“Chocolate ‘vermicelli’ or ‘flakes’”: the product presented in the form of granules or flakes must contain not less than 32% total dry cocoa solids, including not less than 12% cocoa butter and not less than 14% of dry non-fat cocoa solids:

“Chocolate ‘couverture’”: the product must contain not less than 35% total dry cocoa solids, including not less than 31% cocoa butter and not less than 2.5% of dry non-fat cocoa solids:

“Chocolate ‘Gianduja’” (or one of the derivatives of the word ‘gianduja’) nut chocolate: the product must be obtained firstly from chocolate having a minimum total dry cocoa solids content of 32% including a minimum dry non-fat cocoa solids content of 8%, and secondly from finely ground hazelnuts in such quantities that 100 g of the product contain not less than 20 g and not more than 40 g of hazelnuts. The following may be added:

(a) milk and/or dry milk solids obtained by evaporation, in such proportion that the finished product does not contain more than 5% dry milk solids;
(b) almonds, hazelnuts and other nut varieties, either whole or broken, in such quantities that, together with the ground hazelnuts, they do not exceed 60% of the total weight of the product.

### 3. Milk Chocolate, Cream Chocolate, Skimmed Milk Chocolate:

Milk chocolate designates the product obtained from cocoa products, sugars and milk or milk products, which contains:
not less than 25% total dry cocoa solids,
not less than 14% dry milk solids obtained by partly or wholly dehydrating whole milk, semi- or full-skimmed milk, cream, or from partly or wholly dehydrated cream, butter or milk fat,
not less than 2.5% dry non-fat cocoa solids,
not less than 3.5% milk fat,
not less than 25% total fat (cocoa butter and milk fat).

“Milk chocolate ‘vermicelli’ or ‘flakes’”: the product presented in the form of granules or flakes must contain not less than 20% total dry cocoa solids, not less than 12% dry milk solids obtained by partly or wholly dehydrating whole milk, semi- or full-skimmed milk, cream, or from partly or wholly dehydrated cream, butter or milk fat, and not less than 12% total fat (cocoa butter and milk fat).

“Milk chocolate ‘couverture’”: the product must have a minimum total fat (cocoa butter and milk fat) content of 31%.

“Milk chocolate ‘Gianduja’” (or one of the derivatives of the word ‘gianduja’) nut milk chocolate: the product must be obtained firstly from milk chocolate having a minimum content of 10% of dry milk solids, obtained by partly or wholly dehydrating whole milk, semi- or full-skimmed milk, cream or from partly or wholly dehydrated cream, butter or milk fat and secondly from finely ground hazelnuts, in such quantities that 100 g of the product contain not less than 15 g and not more than 40 g of hazelnuts. Almonds, hazelnuts and other nut varieties may also be added, either whole or broken, in such quantities that, together with the ground hazelnuts, they do not exceed 60% of the total weight of the product.

Where in “milk chocolate” the word ‘milk’ is replaced by:
‘cream’: the product must have a minimum milk fat content of 5.5%
‘skimmed milk’: the product must have a milk fat content not greater than 1%.

### 4. Family Milk Chocolate

“Family milk chocolate” designates the product obtained from cocoa products, sugars and milk or milk products and which contains:
not less than 20% total dry cocoa solids,
not less than 20% dry milk solids obtained by partly or wholly dehydrating whole milk, semi- or full skimmed milk, cream, or from partly or wholly dehydrated cream, butter or milk fat,
not less than 2.5% dry non-fat cocoa solids,
not less than 5% milk fat,
not less than 25% total fat (cocoa butter and milk fat).

### 5. White Chocolate

“White chocolate” designates the product obtained from cocoa butter, milk or milk products and sugars which contains not less than 20% cocoa butter and not less than 14% dry milk solids obtained by partly or wholly dehydrating whole milk, semi- or full-skimmed milk, cream, or from partly or wholly dehydrated cream, butter or milk fat, of which not less than 3.5% is milk fat.

### 6. Chocolate a La Taza

“Chocolate a la taza” designates the product obtained from cocoa products, sugars, and flour or starch from wheat, rice or maize, which contains not less than 35% total dry cocoa solids, including not less than 18% cocoa butter and not less than 14% dry non-fat cocoa solids, and not more than 8% flour or starch.

### 7. Chocolate Familiar a La Taza

“Chocolate familiar a la taza” designates the product obtained from cocoa products, sugars, and flour or starch from wheat, rice or maize, which contains not less than 30% total dry cocoa solids, including not less than 18% cocoa butter and not less than 12% dry non-fat cocoa solids, and not more than 18% flour or starch.

The diameter of the chocolate coated capsule of the present invention (i.e. after coating the coating-free capsule with chocolate coating material), i.e. the final capsule which can be placed in the oral cavity and be consumed, is preferably greater than 5 mm, preferably in the range of 6-20 mm, particularly preferentially in the range of 7-18 mm.

The chocolate coating (material) for use in the preparation process according to the present invention preferably comprises or consists of:

90 to 100%, preferably 95 to 100%, of chocolate product, and
0 to 10%, preferably 0 to 5%, of optional constituents.

In case a sugar-free chocolate coating (material) is required, in particular for dietary, e.g. diabetic, purposes, the chocolate coating material may comprise one or more sugar alcohols, preferably selected from the group consisting of erythritol, isomalt, lactitol, maltitol, mannitol, sorbitol, xylitol and hydrogenated starch hydrolysates, the sugar alcohols isomalt, maltitol, mannitol and xylitol being preferred.

The coated capsule according to the present invention in the chocolate coating comprises chocolate (one or more chocolate products) in an amount in the range of 20-95%, preferably in the range of 35-92%, particularly preferentially in the range of 50-92%, based on the total mass of the chocolate coated capsule.

The residual amount of water in the chocolate coating after the chocolate coating process typically is smaller than 2.5%, most typically smaller than 1%, based on the weight of the chocolate coating of the chocolate coated capsule.

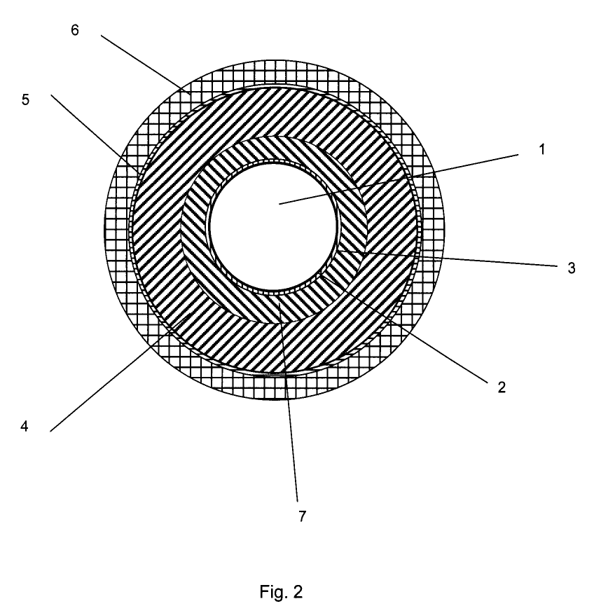

In an (nonpreferential) alternative the chocolate coating (material) may be cast into hemispherical forms of the same size. A single capsule (be it a coating-free capsule, a gummed capsule or a capsule with an inner sugar/sugar alcohol coating (7) as described hereinbelow, see also FIG. 2) is then centered between two chocolate hemispheres (the plane surface of said hemispheres optionally still being supple), said hemispheres subsequently being joined together, e.g. by using an edible binder, preferably chocolate or chocolate coating material, or by (partly) warming the plane chocolate surface and subsequent joining under pressure.

During the preferred chocolate coating process producing a seamless solid chocolate coating, the chocolate coating (material) is usually applied to the coating-free capsule in several layers (chocolate panning), 2 to 200 layers are typical, 5 to 100 layers are preferred. The chocolate coating (material) is usually applied to the coating-free capsule via well-known processes involving coating pan, drum, tumbler or the like. The chocolate coating (material) may, inter alia, be sprayed or cast (manually) onto the coating-free capsules.

For producing preferred seamless chocolate coated capsules of the present invention the chocolate coating material normally is tempered (in a tempering unit) and applied to the coating-free capsule in liquefied or molten form. The temperature of the liquefied or molten chocolate coating material typically lies in the range of 30-65° C., mainly depending on the melting point or range of the chocolate product.

During production of the preferred seamless chocolate coated capsules of the present invention cooling the internal space of the coating unit with a gas stream (e.g. air or an inert gas like nitrogen) is recommended to facilitate hardening/crystallisation of the chocolate coating (material) on the surface of the capsules (be it coating-free capsules or capsules coated with some, but not yet all of the required chocolate layers). The gas stream advantageously has a temperature of 20° C. or below, preferably of 15° C. or below, more preferentially in the range of 0-10° C.

For producing preferred seamless chocolate coated capsules of the present invention the number of chocolate layers can be varied within the amount/proportion limits of chocolate coating given above, depending on the capsule properties to be achieved.

Generally, drying steps concerning the chocolate coated capsules of the present invention should be carried out at temperatures below 25° C., preferably at 20° C. or below in order to avoid remelting and subsequent uncontrolled recrystallisation of the chocolate coating of the chocolate coated capsules.

### (Optional) Constituents of the Chocolate Coating:

If desired, additional flavourings and/or cooling agents, like those mentioned above as constituent of the liquid or viscous core, can be added to the chocolate coating material.

The optionally included one or more flavourings of the chocolate coating may independently from each other be the same or different as the flavourings included in the core liquid. The one or more flavourings included in the chocolate coating and the core liquid preferably are different in those cases in which it is not possible to achieve the desired flavour profile with one or more flavourings in the core liquid alone. The optionally included one or more flavourings of the chocolate coating may independently of each other be in liquid or solid form (at 20° C. and 1013 mbar).

The optionally included one or more flavourings of the chocolate coating advantageously are independently from each other incorporated into the chocolate coating in solid form, i.e. in and/or on a solid edible carrier, preferably in spray-dried form (generally a powder). These solid edible carriers can include proteins, fats, dextrins, waxes, carbohydrates, modified or microcrystalline celluloses, hydrocolloids (such as natural and physically and/or chemically modified or degraded starches), and natural or synthetic gums. Preferred solid edible carriers are gelatine, alginates (preferably sodium alginate), agar, pectin, guar gum, gellan gum, xanthan gum, gum ghatti, tragacanth gum, carrageenan, gum arabic, carboxymethylcellulose, sodium carboxymethylcellulose, milk powder, fat powder, mannitol as well as mixtures of these solid edible carriers. Preferred solid edible carriers for flavourings in spray-dried form are maltodextrins, preferably maltodextrins having a DE-value in the range of 5-28, preferably in the range of 10-20. When flavourings are incorporated into the chocolate coating in spray-dried form, the average particle size of said spray-dried powder preferably is in the range of 10 to 250 microns, more preferably in the range of 20 to 200 microns.

Solid acids such as acids of the monoacid, diacid or triacid type, preferably citric acid, fumaric acid, tartaric acid, succinic acid, adipic acid, malic acid, ascorbic acid, or lactic acid. The use of at least one such acid makes it possible, to tailor the sensory properties of the chocolate coating. Typically the amount of acid(s) in the chocolate coating material is in the range of from 0.1-1.5%.

Color additives applicable in the chocolate coating material are food colors, e.g. FD&C Blue No. 1 (brilliant blue FCF), FD&C Blue No. 2 (Indigotine), FD&C Green No. 3 (Fast Green FCF), FD&C Red No. 3 (Erythosine), FD&C Red No. 40 (Allura red), FD&C Yellow No. 5 (Tartrazine), FD&C Yellow No. 6 (Sunset Yellow FCF), naturally occurring colors like annatto extract, b-apo-8′-carotenal, beta-carotene, beet powder, canthaxanthin, caramel color, carrot oil, cochineal extract (carmine), paprika, paprika oleoresin, riboflavin, saffron, turmeric, turmeric oleoresin, or inorganic pigments, like edible metal oxides, e.g. titanium dioxide.

### (Optional) Constituents Contacting the Chocolate Coating:

To improve the adhesion or bonding of the chocolate coating, in particular of a seamless chocolate coating, to the coating-free capsule, the surface of the coating-free capsule can be roughened or gummed by applying auxiliary materials as an intermediate layer to the coating-free capsule before the chocolate coating process with the chocolate coating material is carried out.

Roughening of the surface of the coating-free capsule usually is achieved by adding corn starch or similar adjuvants in powder form to the wet coating-free capsule before the drying step.

Suitable auxiliary materials for gumming are gum arabic (acacia gum), maltodextrin, starch, sugar (e.g. dextrose), sugar alcohol (e.g. xylitol, isomalt) or gelatine, applied in form of an aqueous solution (the preferred content of auxiliary materials is in the range of 40-75%). Typically the coating-free capsule is coated with one to three layers of auxiliary material before performing the actual coating process with the chocolate coating material.

At this stage and/or when sugar and/or sugar alcohol are applied to the coating-free capsule as auxiliary materials for gumming, (further) layers of sugar and/or sugar alcohol may be applied, thereby creating an inner sugar/sugar alcohol coating as depicted in FIG. 2: after completion of the subsequent chocolate coating step such a capsule comprises an inner sugar/sugar alcohol coating (7) in between the first adhesion layer (3) (first intermediate layer) and the (preferably seamless) chocolate coating (4). The composition of said first adhesion layer (3) (first intermediate layer) and said inner sugar/sugar alcohol coating (7) thus may be identical or different. The total number of layers of said inner sugar/sugar alcohol coating (7) typically lies in the range of 2-40, preferably in the range of 5-25.

If a shiny/glossy final coating is preferred, waxes can be applied as polishing (or glazing) agents to the dry chocolate coated capsules, usually one or two layers of wax (typically 1-5 mL/kg dry chocolate coated capsules) are sufficient.

For sealing the (polished) chocolate coated capsules one or two layers of a sealing agent are advantageous, preferably shellac is employed (typically 1-5 mL/kg dry (polished) chocolate coated capsules).

In addition, the final coating, in particular the sealing agent, can protect the (polished) chocolate coated capsules to some extent from external influences (like humidity, dust, damages during packing process) and prevent the (polished) chocolate coated capsules from sticking to each other and from sticking to the hands of the consumer.

Waxes, mostly refined waxes, like bees wax, carnauba wax or paraffines are either applied in pure (atomised) form or as alcoholic dispersion. Shellac usually is used as an alcoholic (ethanolic) solution.

### Optional Sugar/Sugar Alcohol Coating:

An optional further coating (outer coating) surrounding the chocolate coated capsule is based on solid sugar(s) and/or sugar alcohol(s). The solid sugars or sugar alcohols typically are in crystalline form. Coating the chocolate coated capsule of the present invention with sugar(s) and/or sugar alcohol(s) to some extent protects the chocolate coated capsule from external influences (like humidity, fluctuation in temperature, dust, damages during packing process) and thus improves handling, storability and (mechanical) stability of the sugar/sugar alcohol coated chocolate coated capsules. In addition the solid sugar/sugar alcohol coating gives rise to additional sensorial properties (e.g. like a crunchy feeling when biting/chewing the sugar/sugar alcohol coated chocolate coated capsules) and allows the addition of further flavourings, by incorporation into the solid sugar/sugar alcohol coating.

The diameter of the sugar or sugar alcohol coated chocolate coated capsule of the present invention (i.e. after coating the chocolate coated capsule with sugar(s) and/or sugar alcohol(s)), i.e. the final capsule to be placed in the oral cavity, advantageously is greater than 5.5 mm, preferably in the range of 6.5-22 mm, particularly preferentially in the range of 7.5-20 mm.

The sugar/sugar alcohol coating mixture will normally be applied to the chocolate coated capsule in aqueous form. Typically the sugar(s) or sugar alcohol(s) are used in the coating process as aqueous solutions.

The sugar/sugar alcohol coating mixture for use in a preparation process according to the present invention preferably comprises or consists of:

15 to 45% of water, preferably 20 to 40%
50 to 85% of sugar(s) or sugar alcohol(s), preferably 60 to 80%
0 to 5% of optional constituents

The sugar coating mixture preferably comprises one or more sugars, preferably monosaccharide or disaccharide sugars, preferentially selected from the group consisting of glucose (dextrose), galactose, arabinose, fructose, lactose, sucrose (saccharose) and maltose, the sugars glucose, fructose and sucrose being preferred.

The sugar alcohol coating mixture preferably comprises one or more sugar alcohols, preferably selected from the group consisting of erythritol, isomalt, lactitol, maltitol, mannitol, sorbitol, xylitol and hydrogenated starch hydrolysates, the sugar alcohols isomalt, sorbitol, maltitol, mannitol and xylitol being preferred.

Preferably the sugar/sugar alcohol coating of the chocolate coated capsule according to the present invention comprises one or more sugar(s) or sugar alcohol(s) in an amount in the range of 5-60%, more preferably in the range of 10-50%, based on the total mass of the sugar/sugar alcohol coated chocolate coated capsule.

The residual amount of water in the sugar/sugar alcohol coating after the coating process typically is smaller than 2%, most typically smaller than 1%, based on the weight of the sugar/sugar alcohol coating of the capsule.

The sugar/sugar alcohol coating mixture is usually applied to the chocolate coating capsule via well-known panning or dragee coating processes, involving dragee pan, coating drum, tumbler or the like.

During the sugar/sugar alcohol coating process, the coating is applied to the chocolate coated capsule in several layers, 2 to 100 layers are typical. If the chocolate coated capsule is coated mainly or exclusively with sugar(s), 5 to 40 layers are preferred, since sugar coatings generally exhibit a greater hardness than those with sugar alcohols. In case the chocolate coated capsule is coated mainly or exclusively with sugar alcohol(s), 10 to 80 layers are preferred.

The number of layers can be varied within the limits given above, depending on the capsule properties to be achieved.

### (Optional) Constituents of the Sugar/Sugar Alcohol Coating:

If desired, flavourings and/or cooling agents, like those mentioned above as constituent of the liquid or viscous core, can be added to the coating mixture. Furthermore, breath control actives, antitussive actives and/or oral anesthetic actives can be added.

Solid acids such as acids of the monoacid, diacid or triacid type, preferably citric acid, fumaric acid, tartaric acid, succinic acid, adipic acid, malic acid, ascorbic acid, or lactic acid. The use of at least one such acid makes it possible to tailor its sensory properties. Typically the amount of acid(s) in the sugar/sugar alcohol coating mixture is in the range of 0.25-1.5%.

Colourants or dyes applicable in the coating typically are water-soluble food colourants, e.g. FD&C Blue No. 5, FD&C Yellow No. 1, brilliant blue, allura red, or inorganic pigments, like edible metal oxides, e.g. titanium dioxide.

### (Optional) Constituents Contacting the Sugar/Sugar Alcohol Coating:

To improve the adhesion or bonding of the sugar/sugar alcohol coating to the chocolate coated capsule, the surface of the chocolate coated capsule can be roughened or auxiliary materials (so-called gumming materials) can be applied as an intermediate layer to the chocolate coated capsule before the coating process with the sugar/sugar alcohol coating materials is carried out.

Roughening of the surface of the chocolate coated capsule usually is achieved by adding corn starch or similar adjuvants in powder form to the wet coating-free capsule before the drying step.

Suitable auxiliary materials for gumming are gum arabic (acacia gum), maltodextrin, starch, sugar (e.g. dextrose), sugar alcohol (e.g. xylitol, isomalt) or gelatine, applied in form of an aqueous solution (the content of auxiliary materials is in the range of 40-75%). Typically the chocolate coated capsule is coated with one to three layers of auxiliary material before performing the actual coating process with sugar/sugar alcohol coating materials.

In addition, during application of the auxiliary material to the chocolate-coated capsule, it is possible to add actives to the aqueous solution auxiliary material. For example, suitable actives are the breath control actives, antitussive actives or oral anesthetic actives given above.

If a shiny/glossy final coating on the sugar/sugar alcohol coating is preferred, waxes can be applied as polishing (or glazing) agents to the dry sugar/sugar alcohol coating of the chocolate coated capsules, usually one or two layers of wax (typically 1-5 mL/kg dry (polished) sugar/sugar alcohol coated chocolate coated capsules) are sufficient.

For sealing the (polished) sugar/sugar alcohol coating of the chocolate coated capsules one or two layers of a sealing agent are advantageous, preferably shellac is employed (typically 1-5 mL/kg dry (polished) sugar/sugar alcohol coated chocolate coated capsules).

In addition, the final coating, in particular the sealing agent, can protect the (polished) sugar/sugar alcohol coated chocolate coated capsules to some extent from external influences (like humidity, dust, damages during packing process) and prevent the (polished) sugar/sugar alcohol coated chocolate coated capsules from sticking to each other and from sticking to the hands of the consumer.

Waxes, mostly refined waxes, like bees wax, carnauba wax or paraffines are either applied in pure (atomised) form or as alcoholic dispersion. Shellac usually is used as an alcoholic solution.

The capsules comprising an inner chocolate coating and an outer sugar/sugar alcohol coating can be prepared by a method comprising the following steps:
preparing a coating-free capsule—having (i) a liquid or viscous core and (ii) a solid shell surrounding this core,
chocolate coating the coating-free capsule, optionally only after applying an intermediate layer or intermediate layers to the coating-free capsule,
sugar/sugar alcohol coating the resulting chocolate coated capsule, optionally only after applying on intermediate layer or intermediate layers to the chocolate coated capsule, and
optionally polishing and/or sealing the sugar/sugar alcohol coated chocolate coated capsule.

Preferably the sugar/sugar alcohol coating is applied to a dried chocolate coated capsule.

When the capsules comprising an inner chocolate coating and an outer sugar/sugar alcohol coating shell comprise a seamless coating-free capsule, they can be prepared by a corresponding method comprising the following steps:
pumping a liquid or viscous core material and a gelatine or alginate-containing curable shell mixture simultaneously through a concentric multi-component nozzle so that they drip into a cooling liquid with the formation of a capsule, (i.e. forming a capsule upon immersion into the cooling liquid),
drying said capsule,
chocolate coating the resulting dried capsule (hereinafter also: coating-free capsule), optionally only after applying an intermediate layer or intermediate layers to the dried capsule,
sugar/sugar alcohol coating the resulting chocolate coated capsule, optionally only after applying an intermediate layer or intermediate layers to the chocolate coated capsule, and
optionally polishing and/or sealing the sugar/sugar alcohol coated chocolate coated capsule.

### Appearance:

Capsules according to the invention are ball-shaped (spherical).

The ratio between the largest and the smallest diameter of a spherical coated capsule according to the invention is not more than 1.2, preferably not more than 1.1.

Preferred embodiments of the spherical coated capsules of the present invention are shown in the attached drawings:

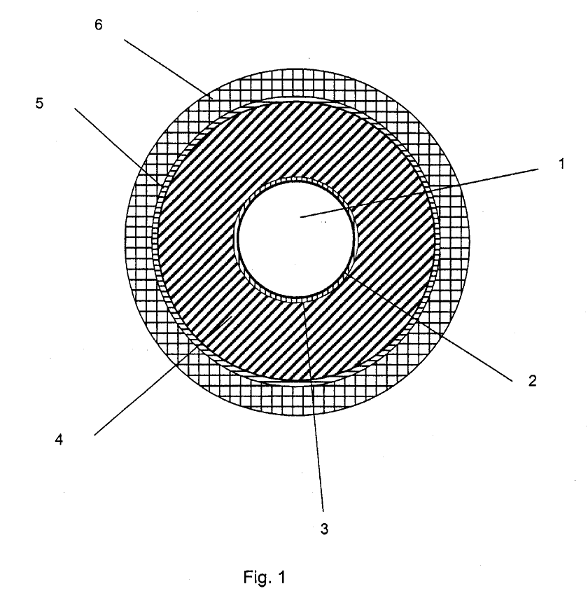

FIG. 1 is a schematic cross section (not according to scale) of a chocolate coated spherical capsule according to the present invention. The capsule comprises a liquid or viscous core (1), a seamless shell (2) surrounding said core, a first adhesion layer (3) (first intermediate layer), a seamless chocolate coating (4), a second adhesion layer (5) (second intermediate layer), and an outer sugar/sugar alcohol coating (6).

FIG. 2 shows a schematic cross section (not according to scale) of an alternative embodiment of a capsule according to the present invention. The alternative embodiment according to FIG. 2 is similar to the embodiment of FIG. 1, but comprises an inner sugar/sugar alcohol coating (7) in between the first adhesion layer (3) (first intermediate layer) and the chocolate coating (4).

Regarding the ingredients of the respective cores, layers and coatings of the embodiments according to FIGS. 1 and 2 it is referred to the description above and the examples below.

### EXAMPLES

Preferred embodiments of the invention are explained in more detail below on the basis of examples.

### Examples 1-7

### Method for the Preparation of Coating-Free Capsules (Used in the Preparation of Coated Capsules According to the Invention)—General Procedure (Multi-Component Nozzle Method with Immersed Nozzle)

The constituents indicated in the appended table “Examples 1-7” for the shell mixture are added together and heated to 80° C. in a water bath until a clear solution that is essentially free from air bubbles has formed. Preferably solutions with solid contents of 20-40% (m/m) are used.

The core liquid is prepared at 10-20° C.

Shell liquid and core liquid are fed via a pump system to a concentric two-component nozzle. The line for the shell liquid is kept at 60-80° C. during this operation. The concentric two-component nozzle dips into a liquid bath that is filled with vegetable oil. The temperature of this oil bath is approximately 14° C.

With support from additional vibrational stimulus of the liquid, the liquid jet issuing from the nozzle into the oil bath disintegrates into individual droplets which are seamless coating-free capsules consisting of core and shell.

Adhering oil is removed from the capsules by centrifuging while the coating-free capsules are still wet and the capsules are then dried with continuous motion in a dry stream of air. Conventional vortex dryers or drum dryers can be used. The prerequisite for a good drying result is that the capsules can be kept in motion by rotation or by turbulent air. In some cases it is advisable to use an anticaking agent for this purpose.

However, in most cases the use of an anticaking agent is not desired. Specifically, a transparent and glossy shell is obtained if the composition of the shell mixture is so chosen that it is possible to dispense with the use of an anticaking agent such as, for example, silica during drying and there is nevertheless no sticking of the coating-free capsules.

### Examples 1-7

Composition and consumption characteristics of the dried coating-free capsules/*Note: Adhering residual water depends on the atmospheric humidity and can therefore vary.

| | Diameter | | Shell | | | | |
|---|---|---|---|---|---|---|---|
| | of coating- | | thickness/ | | | | |
| | free | Shell | capsule | Shell | Core | | |
| | capsule | thickness | diameter | content | content | | |
| No. | [mm] | [μm] | ratio | [% (m/m)] | [% (m/m)] | Composition of shell | Composition of core |
| 1 | 4.5 | 45 | 0.010 | 7.5 | 92.5 | 78% pig gelatine 260 Bloom | 30% peppermint flavouring |
| | | | | | | 17% glycerol | 70% vegetable oil |
| | | | | | | 5% water* | |
| 2 | 6 | 72 | 0.012 | 9 | 91 | 70% pig gelatine 260 Bloom | 30% orange oil flavouring |
| | | | | | | 20% glycerol | 70% vegetable oil |
| | | | | | | 8.9% water* | |
| | | | | | | 0.5% aspartame | |
| | | | | | | 0.5% acesulfame K | |
| | | | | | | 0.1% allura red and FD&C Yellow #1 | |
| 3 | 7.5 | 75 | 0.010 | 6 | 94 | 72% pig gelatine 275 Bloom | 25% wintergreen flavouring |
| | | | | | | 18% glycerol | 75% vegetable oil |
| | | | | | | 10% water* | |
| 4 | 5.5 | 50 | 0.009 | 10 | 90 | 80% pig gelatine 260 Bloom | 15% peppermint flavouring |
| | | | | | | 10% Sorbitol | 85% vegetable oil |
| | | | | | | 10% water* | |
| 5 | 6.5 | 54 | 0.008 | 5 | 95 | 72% cattle gelatine 280 Bloom | 30% cinnamon flavouring |
| | | | | | | 20% glycerol | 70% vegetable oil |
| | | | | | | 7.9% water* | |
| | | | | | | 0.1% allura red | |
| 6 | 8 | 96 | 0.012 | 9 | 91 | 70% pig gelatine 260 Bloom | 65% mint flavouring |
| | | | | | | 20% glycerol | 35% vegetable oil |
| | | | | | | 7.9% water* | |
| | | | | | | 2% sucralose | |
| | | | | | | 0.1% brilliant blue | |
| 7 | 5 | 72 | 0.014 | 11 | 89 | 74% fish gelatine 165 Bloom | 50% mint flavouring |
| | | | | | | 16% glycerol | 50% vegetable oil |
| | | | | | | 7.9% water* | |
| | | | | | | 2% gellan gum KELCOGEL F | |
| | | | | | | 2% sucralose | |
| | | | | | | 0.1% brilliant blue | |

### Table: Gel Points

| Composition of the aqueous shell | Gel | Viscosity | |
|---|---|---|---|
| solution | point | at 80° C. | Flexibility* |
| 75% water | 34° C. | 61 mPas | low |
| 20% cattle gelatine 240 Bloom | | | |
| 5% glycerol | | | |
| 75% water | 35° C. | 71 mPas | low |
| 20% chicken gelatine 250 Bloom | | | |
| 5% glycerol | | | |
| 75% water | 30° C. | 130 mPas | moderate |
| 10% pig gelatine 260 Bloom | | | |
| 10% fish gelatine 110 Bloom | | | |
| 5% glycerol | | | |
| 75% water | 29° C. | 90 mPas | high |
| 15% pig gelatine 260 Bloom | | | |
| 5% fish gelatine 0 Bloom | | | |
| 5% glycerol | | | |
| 75% water | 32° C. | 85 mPas | high |
| 15% chicken gelatine 250 Bloom | | | |
| 5% fish gelatine 0 Bloom | | | |
| 5% glycerol | | | |

Flexibility*: Assessment on bending over a 200 μm thick film which had been produced from the shell solution by pouring out and drying in air (20° C., 40% relative atmospheric humidity, at least 20 h).

### Examples 8

### Coating-Free Capsules

As described for the examples 1-7 above, coating-free capsules with a diameter of 7.3 mm are prepared, having the following composition:

### Core: 940 Parts by Weight of the Coating Free Capsule

| Core ingredient | % by weight |
|---|---|
| fractioned Coconut oil | 58.04 |
| Peppermint oil (Mentha arvensis) | 28.8 |
| D-Limonene | 2.5 |
| Methyl salicylate | 0.5 |
| Vanillin | 1.0 |
| Clove bud oil | 0.7 |
| Neotame | 0.02 |
| Aspartame | 0.04 |
| Sucralose | 1.2 |
| Pepper oil and Piperine | 0.1 |
| Frescolat ® ML | 2.5 |
| Frescolat ® MPC | 1.4 |
| Diethyl tartrate | 2.2 |
| Ethanol | 0.7 |
| Vitamin E - acetate | 0.3 |

Frescolat ® ML = menthyl lactate (Symrise GmbH & Co. KG)
Frescolat ® MPC = menthol propyleneglycol carbonate (Symrise GmbH & Co. KG)

### Shell: 60 Parts by Weight of the Coating Free Capsule

| Shell ingredient | % by weight |
|---|---|
| Water | 2.9 |
| Glycerol | 20.3 |
| Gelatine 280 Bloom | 76.1 |
| Sucralose | 0.6 |
| FD&C Blue #5 | 0.1 |

The coating-free capsules have the following properties

7.3 mm diameter; 6% shell, 94% core liquid, shell thickness: about 74 μm
weight per coating-free capsule: 193 mg

### Example 9

### Method for the Preparation of Chocolate Coated Capsules According to the Invention

500 g of the coating-free capsules of example 8 are placed in a rotating chocolate coating pan. All chocolate coating steps are carried out in this coating unit.

A gumming mixture is prepared consisting of 5 g of a 50% solution of gum arabic, 5 g of dextrose (70% in water) and 0.5 g of peppermint oil (mentha piperita). To improve the adhesion of the chocolate coating to be applied in the next step, one layer of this gumming mixture is first applied to the coating-free capsules of example 8 and a second gumming layer is thereafter applied in the form of xylitol dust (25 g of xylitol based QUICK COAT, a product of Alfred L. Wolff GmbH, Germany). These capsules are then placed on trays and dried in a drying chamber at 20° C. and a relative humidity of below 40%, the airstream having a velocity of 1 to 5 m/s.

A total of 1620 g of commercially available dark chocolate [(“Schwarze Herrenschokolade”, Stollwerck GmbH, Germany, more than 60% of total dry cocoa solids, optionally adding 0.5 g vanillin and/or 1.0 g powder or extract of chili (capsicum frutescens, includes capsaicin)] is applied in multiple layers onto the dried gummed capsules. During the chocolate coating process the internal space of the coating unit was cooled with an airstream having a temperature of 9° C.

Optionally the obtained chocolate coated capsules are dried in a drying chamber at 15° C. and a relative humidity of below 30%, the airstream having a velocity of 1 to 5 m/s.

The coating process may be stopped at this point of the process, the chocolate coated capsules now have the desired properties, which are as follows:

about 10 mm diameter, about 76% of the total capsule weight is chocolate used in the coating.

Optionally, a shiny final layer of carnauba wax and a sealing with shellac can be added to the chocolate coated capsules.

### Example 10

### Method for Sugar Coating the Chocolate Coated Capsules According to the Invention

Depending on the properties of the chocolate coated capsules, e.g. hardness and roughness of the chocolate coating, a second gumming step may be recommendable before applying the sugar coating to the chocolate coated capsules.

The chocolate coated capsules of example 9 (without shiny final layer) are placed in a panning machine (DSG 20V/10, Acosystems Prozesstechnologie GmbH, Delligsen, Germany). All following coating steps employing sugar are carried out in this panning machine.

A gumming mixture is prepared consisting of a 50% solution of gum arabic, and an 70% aqueous solution of dextrose. To improve the adhesion of the sugar coating to be applied in the next step, one or two layers of this gumming mixture are first applied to the chocolate coated capsules (15 mL of gumming mixture per kg of chocolate coated capsules). Thereafter a further gumming layer is applied in the form of dextrose dust (70 g of dextrose based QUICK COAT, a product of Alfred L. Wolff GmbH, Germany).

In the main sugar coating step, the gummed chocolate coated capsules are then coated with 18 layers using 1420 g of a sugar coating mixture consisting of 68.5% dextrose, 1.1% titanium dioxide and 30.4% of water. The capsules have a bright white appearance and a smooth surface. It should be noted that almost all the water introduced as part of the sugar coating mixture evaporates during the sugar coating process.

In the next step, the resulting capsules are then coated with 3 layers using the following coating mixture: 68% dextrose, 2% of an aqueous solution of a mixture of FD&C Blue #1 and D&C Red #33, and 30% water. The capsules now have a smoother surface and a purple color.

In order to remove residual amounts of water from the coating, the resulting capsules are then placed on trays and dried for 8-12 hours in a drying chamber at a temperature of 18° C. and a relative humidity of below 40%, the airstream having a velocity of 1 to 5 m/s.

The final sugar coated chocolate coated capsules have the following properties:

about 12 mm diameter, about 31% of the total capsule weight is dextrose (sugar) used in the outer coating, about 51.5% of the total capsule weight is chocolate used in the chocolate coating.

Finally shiny layers (1.5 mL per kg capsules) of waxes (QUICK GLANZ H, a liquid alcoholic dispersion of refined waxes, a product of Alfred L. Wolff GmbH, Germany) and sealing layers (1.5 mL per kg capsules) with shellac (QUICK LAC, an alcoholic solution of shellac, a product of Alfred L. Wolff GmbH, Germany) are applied to the sugar coated chocolate coated capsules.

### Example 11

### Method for the Preparation of Sugar-Free Chocolate Coated Capsules According to the Invention

500 g of the coating-free capsules of example 8 are placed in a rotating chocolate panning unit. All chocolate coating steps are carried out in this unit.

All further steps are carried out as described in example 9 whereby dextrose is replaced by xylitol and a total of 1620 g of commercially available sugar-free isomalt milk chocolate is used instead of dark chocolate.

The resulting chocolate coated capsules are sugar-free and essentially have the same appearance as the capsules produced according to example 9.

### Example 12

### Method for Sugar Alcohol Coating the Sugar-Free Chocolate Coated Capsules According to the Invention

The chocolate coated capsules of example 11 (without shiny final layer) are placed in a panning machine (DSG 20V/10, Acosystems Prozesstechnologie GmbH, Delligsen, Germany). All following coating steps employing sugar alcohols are carried out in this panning machine.

All further steps are carried out as described in example 10 whereby dextrose is replaced by a 1:1 (m/m) mixture of isomalt and xylitol.

The resulting sugar alcohol coated chocolate coated capsules are sugar-free and essentially have the same appearance as the capsules produced according to example 10.

### Example 13

### Method for the Preparation of Chocolate Coated Capsules According to the Invention

500 g of the coating-free capsules of example 2 are placed in a rotating chocolate coating pan. All chocolate coating steps are carried out in this coating unit.

All further steps are carried out as described in example 9 whereby a total of 1800 g of milk chocolate with caramelised walnuts is used instead of dark chocolate. The chocolate used is commercially available milk chocolate with caramelised walnuts (Lindt Excellence, Chocoladefabriken Lindt & Sprüngli AG, Switzerland, more than 30% of total dry cocoa solids).

The coating process may be stopped at this point of the process, the chocolate coated capsules now have the desired properties, which are as follows:

about 9.5 mm diameter, about 83% of the total capsule weight is chocolate (including caramelised walnuts) used in the coating.

### Examples 14

### Coating-Free Capsules with Coffee Flavour

As described for the examples 1-7 above, coating-free capsules with a diameter of 7.0 mm are prepared, having the following composition:

### Core: 940 Parts by Weight of the Coating Free Capsule

| Core ingredient | % by weight |
|---|---|
| fractioned coconut oil | 94.84 |
| Oil soluble coffee bean extract | 0.50 |
| Coffee-flavouring V* | 1.00 |
| Sucralose | 0.20 |
| Triacetin (glycerol triacetate) | 0.90 |
| Ethanol | 2.46 |
| Water | 0.10 |

Coffee-flavouring V* consists of triacetin (glycerol triacetate); 1,2-propylene glycol; 2,3-methylfuranthiol; furfuryl thioacetate; difurfuryl disulfide; 3,3-methylthiobutyl formate; 2,6-dimethoxy phenol; 2-acetylthiazol; 2-methylbutanal; 3-methylbutanal; 2,3-butandione; 2,3-methylethyl pyrazine; 2,3-methoxymethylpyrazine; 3,5,2-methyldiethyl pyrazine; 2-ethyl-3,5-dimethylpyrazine; 2-ethyl-3,6-dimethylpyrazine; 2-ethyl-3,5-dimethyl pyrazine; 3-ethyl-2,5-dimethylpyrazine; 2-methoxy-3-(1-methylpropyl) pyrazine; 4-vinylguaiacol; 4-ethylguaiacol; 2,5-dimethyl-4-hydroxy-3(2H)-furanone; 4-methyl-5-ethyl-3-hydroxy-2(5H)-furanone; acetaldehyde; beta-damascenone; 2,3-pentandione; ethyl acetate; acetic acid, butyric acid; caproic acid; indole and dl-alpha-tocopherole.

### Shell: 60 Parts by Weight of the Coating Free Capsule

| Shell ingredient | % by weight |
|---|---|
| Glycerol | 20.37 |
| Gelatine 280 Bloom | 78.49 |
| Aqueous coffee-extract | 1.13 |
| (caffeine - content 1.9 wt %) | |

The coating-free capsules have the following properties:

7.0 mm diameter; 6% shell (5.42% solids, 0.58% water), 94% core liquid, weight per coating-free capsule: 172 mg
Caffeine—content (HPLC-analysis): 6 ppm, corresponding to approx. 0.001 mg caffeine per capsule.

### Example 15

### Method for the Preparation of Chocolate Coated Capsules According to the Invention

940 g of the coating-free capsules of example 14 are placed in a panning machine (DSG 20V/10, Acosystems Prozesstechnologie GmbH, Delligsen, Germany). All following coating steps employing sugar are carried out in this panning machine.

A gumming mixture is prepared consisting of a 50% solution of gum arabic, glucose syrup (DE 40) and an 72% aqueous solution of dextrose. To improve the adhesion of the sugar coating to be applied in the next step, two layers of this gumming mixture first are applied to the coating-free capsules of example 14 (14 g of gumming mixture per kg of coating-free capsules). Thereafter a further gumming layer is applied in the form of dextrose dust (35 g of dextrose based QUICK COAT, a product of Alfred L. Wolff GmbH, Germany). After subsequent drying on trays in a drying chamber at 20° C. for 30 minutes, 970 g of gummed coating-free capsules are obtained.

In the main sugar coating step, the gummed coating-free capsules are then coated with 30 layers using a sugar coating mixture consisting of 74.0% sucrose and 26.0% of water. After subsequent thorough drying at 20° C. on trays in a drying chamber, 1500 g of sugar-coated capsules are obtained.

In the next step, the thus obtained 1500 g of sugar-coated capsules are placed in a rotating chocolate coating pan. All chocolate coating steps are carried out in this chocolate coating unit.

A homogenous mixture consisting of 49% of commercially available dark chocolate (Barry Callebaut Type 811), 49% milk chocolate (Barry Callebaut Type 668) and 2.0% of a spray-dried flavouring is applied in multiple layers onto the dried gummed capsules until a capsule diameter of 11 mm is reached. The spray-dried flavouring used as part of the chocolate coating comprises an aqueous coffee-extract (having a caffeine-content of 4.8% according to HPLC-analysis) and an almond-caramel flavouring. The solid edible carrier of the spray-dried flavouring is a 80/20 (m/m) mixture of maltodextrin (DE 18-20) and gum arabic. During the chocolate coating process the internal space of the coating unit is cooled with an airstream having a temperature of 9° C.

The resulting capsules are dried in a drying chamber at 15° C. and a relative humidity of below 30%, the airstream having a velocity of 1 to 5 m/s yielding 4675 g of chocolate coated capsules having the desired properties, which are as follows:

11 mm diameter, about 11.4% of the total capsule weight is sucrose used in the sugar coating and about 67.9% of the total capsule weight is chocolate used in the chocolate coating (including the spray-dried flavouring used in the chocolate coating).

Capsule weight: 850 mg per capsule; caffeine-content of the chocolate coated capsules: about 500 ppm, corresponding to about 0.425 mg caffeine per capsule.

Finally shiny layers (2.5 mL per kg capsules) of waxes (QUICK SHINE, a product of Alfred L. Wolff GmbH, Germany) and sealing layers (1.5 mL per kg capsules) with shellac (QUICK LAC, an alcoholic solution of shellac, a product of Alfred L. Wolff GmbH, Germany) are applied to the chocolate coated capsules.

1-10. (canceled)

11. A spherical coated capsule comprising:
(a) a coating-free capsule comprising (i) a liquid or viscous core (1) and (ii) a solid shell (2) surrounding said liquid or viscous core (1); and
(b) a solid chocolate coating (4) surrounding said coating-free capsule; wherein:
the diameter of said spherical coated capsule is in the range of from 4 to 20 mm;
said solid chocolate coating (4) comprises chocolate in an amount in the range of from 20 to 95% (m/m), based on the total mass of the spherical coated capsule;
the diameter of said coating-free capsule is in the range of from 3 to 10 mm;
the thickness of said solid shell (2) of said coating-free capsule is in the range of from 20 to 200 μm;
the ratio of the thickness of said solid shell (2) to the diameter of said coating-free capsule is in the range of from 0.004 to 0.04;
said solid shell (2) comprises from 70 to 90% (m/m) gelatine or alginate and from 10 to 30% (m/m) plasticiser, based on the dry mass of said solid shell; and
said liquid or viscous core (1) comprises a flavouring content in the range of from 1 to 100% (m/m), based on the total mass of the core.

12. The spherical coated capsule of claim 11, wherein said solid shell (2) is seamless.

13. The spherical coated capsule of claim 11, wherein said solid chocolate coating (4) is seamless.

14. The spherical coated capsule of claim 11, further comprising:
one or more first intermediate layers (3) arranged between said solid shell (2) and
said solid chocolate coating (4).

15. The spherical coated capsule of claim 11, further comprising:
(c) a coating (6) comprising solid sugar(s) and/or sugar alcohol(s) surrounding said spherical coated capsule.

16. The spherical coated capsule of claim 15, further comprising:
one or more second intermediate layers (5) arranged between said solid chocolate coating (4) and said coating (6).

17. The spherical coated capsule of claim 11, wherein:
the diameter of said coating-free capsule is in the range of from 4 to 9 mm;
the thickness of said solid shell (2) is in the range of from 30 to 150 μm; and
the ratio of the thickness of said solid shell (2) to the diameter of said coating-free capsule is in the range of from 0.006 to 0.025.

18. The spherical coated capsule of claim 17, wherein:
the diameter of said coating-free capsule is in the range of from 4.5 to 8.5 mm;
the thickness of said solid shell (2) is in the range of from 40 to 120 μm; and
the ratio of the thickness of said solid shell (2) to the diameter of said coating-free capsule is in the range of from 0.008 to 0.02.

19. A method for preparing a spherical coated capsule, comprising:
preparing a coating-free capsule comprising (i) a liquid or viscous core (1) and (ii) a solid shell (2) surrounding said liquid or viscous core (1);
coating said coating-free capsule with a chocolate coating (4), optionally after applying one or more first intermediate layers (3) to said coating-free capsule to form a chocolate coated capsule; and
optionally polishing and/or sealing said chocolate coating (4).

20. The method of claim 19, wherein the preparation of said coating-free capsule comprises:
pumping a liquid or viscous core material and a gelatine or alginate-containing curable shell mixture simultaneously through a concentric multi-component nozzle so that they drip into a cooling liquid to form a wet capsule; and
drying said wet capsule.

21. The method of claim 19, further comprising:
coating said chocolate coated capsule with a coating material comprising solid sugar(s) and/or sugar alcohol(s).
